(19) United States
(12) Patent Application Publication
(10) Pub. No.: US 2022/0037004 A1
(43) Pub. Date: Feb. 3, 2022

(54) HEALTHCARE WORKER BURNOUT DETECTION TOOL

(71) Applicant: Hennepin Healthcare System, Inc., Minneapolis, MN (US)

(72) Inventor: Deepti Pandita, Minneapolis, MN (US)

(21) Appl. No.: 17/389,814

(22) Filed: Jul. 30, 2021

Related U.S. Application Data
(60) Provisional application No. 63/059,578, filed on Jul. 31, 2020.

(51) Int. Cl. G16H 40/20; G16H 10/20

(52) U.S. Cl. CPC G16H 40/20; G16H 10/20

(57) ABSTRACT

Described embodiments include an application configured to detect burnout of healthcare workers. The application receives a request from a mood survey application database server to initiate a mood survey, and in response to receipt of the notification, causes a notification to be provided to a user via a display including the request to perform the mood survey. In response to selection of the notification by the user, the application causes the mood survey to be presented to the user via the display. In response to completion of the survey by the user, the application provides mood survey results associated with the mood survey to the mood server database. The application further causes burnout mitigation resources received from the mood survey application database server to be presented to the user via the display, wherein the burnout mitigation resources are received based on the mood survey results.

### CROSS-REFERENCE TO RELATED APPLICATION

This patent application claims the priority of U.S. Provisional Patent Application No. 63/059,578, filed Jul. 31, 2021, entitled “Healthcare Worker Burnout Application,” the disclosure content of which is hereby incorporated by reference.

### BACKGROUND

In many circumstances, healthcare practitioners and clinicians work under extremely stressful and demanding conditions with very little opportunity to physically, mentally, and emotionally recover during or between long shifts, which can lead to physical, mental, and/or emotional burnout. Conventional methods used to detect burnout are often unreliable, usually because participation is time-consuming and inconvenient. In addition, the current burnout assessment methodology tends to be based on standardized or customized surveys administered to clinicians by health care systems or other third party providers when the healthcare worker is no longer in the middle of a stressful situation or shift.

### BRIEF DESCRIPTION OF THE DRAWINGS

FIG. 1 depicts various exemplary user interface screenshot layouts for the application hosted on a mobile device in accordance with embodiments of the disclosure.

FIG. 2 depicts a block diagram of an exemplary system to support the application hosted on the mobile device, in accordance with embodiments of the disclosure.

FIG. 3 depicts a block diagram of components of a computing device in accordance with an embodiment of the present disclosure.

### DETAILED DESCRIPTION

Certain details are set forth below to provide a sufficient understanding of embodiments of the disclosure. It will be clear to one skilled in the art, however, that embodiments of the disclosure may be practiced without various aspects of these particular details. In some instances, well-known circuits, control signals, timing protocols, computer system components, and software operations have not been shown in detail in order to avoid unnecessarily obscuring the described embodiments of the disclosure.

This disclosure describes embodiments of an application hosted on a client, such as a mobile device (e.g., tablet, mobile phone, or any other handheld mobile computing device) or a web browser that is configured to detect burnout of healthcare workers, (e.g., providers, clinicians, fellows, residents, nurses, support staff, students and learners in the healthcare fields, etc.). In many circumstances, healthcare practitioners and clinicians work under extremely stressful and demanding conditions with very little opportunity to physically, mentally, and emotionally recover during or between long shifts, which can lead to physical, mental, and/or emotional burnout. Currently, there is no proven/reliable method or system capable of detecting healthcare worker burnout in real-time, and accordingly, no way to preventatively help mitigate the burnout until it's too late. Existing methodologies for detecting healthcare worker burnout include email-based surveys or web-based surveys that are often unreliably taken and/or taken at a time and place convenient for the healthcare worker. That is, the current burnout assessment methodology tends to be based on standardized or customized surveys administered to clinicians by health care systems or other third party providers when the healthcare worker is no longer in the middle of a stressful situation or shift.

Accordingly, the application may cause notifications to randomly appear on the healthcare worker's mobile phone prompting them to perform a brief survey to gauge their current emotional state. The application may cause notifications to appear on the healthcare worker's mobile phone at preset times as determined by the unique needs of the practice, the instructor, or the situation, prompting them to perform a brief survey to gauge their current emotional state. For example, during periods of crisis or surge, this application may be useful to gauge stress level of high volume areas such as intensive care units or emergency rooms. The results of the surveys may be provided to a backend application that is configured to analyze and aggregate survey results, and provide morale information/metrics across various groups or sub-groups. The groups may be divided by location, organization, practice, or department (e.g., across a hospital system, within a single hospital, within a single department, or a combination thereof), by role (e.g., doctor, nurse, other staff), or any combination thereof. Causing the surveys to be performed at random times or at customized preset times as determined by the practice or instructor, or to be triggered for deployment during crisis or surge situations, and having access to real-time analysis of survey results will allow administrators to detect low morale “hot spots” in real time, which can then be monitored and steps can be taken to mitigate burnout using “just in time” interventions. One main goal of the application is to capture sentiment of how a healthcare worker feels throughout the week using emoji-based responses. To engage users, the application is built as a mood tracker, so users are able to track their emotional progress over time, giving them intrinsic motivation to continue using the application.

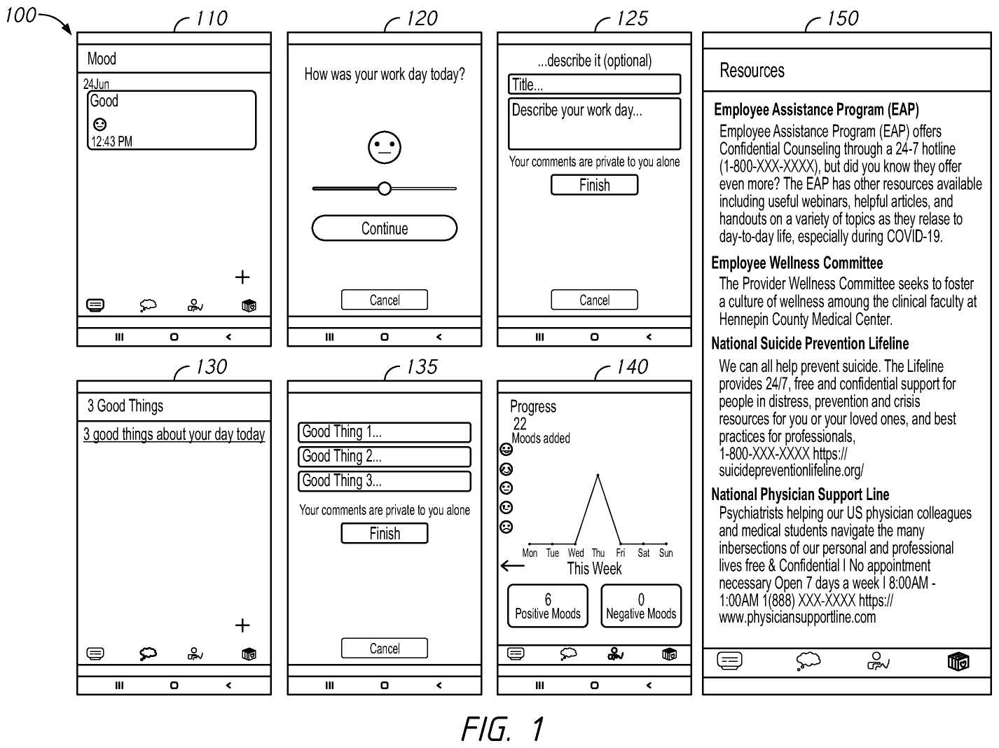

FIG. 1 depicts various exemplary user interface screenshot layouts 100 for the application hosted on a mobile device, in accordance with embodiments of the disclosure. In other embodiments, the application may be accessed via a web browser. As shown in the first screenshot 110, the application may include a “mood” journal that depicts a list of previous “mood” survey responses and comments (e.g., “moods”), along with a selection icon to add a new “mood”.

As shown in the second screenshot 120, the application may include a “mood” survey screen. The mood survey screen may include a mood scale represented by selectable emojis, along with a text field to enter any relevant comments at the time the survey is being taken (as shown in the third screenshot 125). In some examples, additionally or alternatively to the text field, the mood survey may further include common responses to describe a person's day. In some examples, the common responses may be selectable from a list, including both positive and negative responses. In other examples, rather than an emoji-based scale, the application may use a number-based scale, an adjective-based scale (e.g., happy, sad, content, etc.), or any combination thereof (including the emoji-based scale). It is appreciated that the scale may extend vertically on the screen, rather than horizontally, or the scale may appear as a dial, without departing from the scope of the disclosure.

As shown in the fourth screenshot 130, the application may further include a “good things” page. The “good things” page may include positive past comments made by or about (from other users) the user. The user may add up to a certain number of good things, such as three good things as shown in the fifth screenshot 135. More or fewer than three good things may be added without departing from the scope of the disclosure.

As shown in the sixth screenshot 140, the application may include a mood survey progress page, which includes a streak of days in which a mood survey has been taken, a trend line indicating changes in mood survey responses over time, an indication of a number of times each survey response has been entered over some period of time (e.g., all time, per shift, daily, weekly, monthly, quarterly, yearly, etc.).

As shown in the seventh screenshot 150, the application may include a resource page that provides links to various resources to help mitigate physical burnout, such as links to helplines or other resources to seek help when a healthcare worker feels they need it, links to resources (e.g., other applications or web pages) that include information to guide a user through various activities or exercises, or any combination thereof.

In some examples, the application may be developed to support many different mobile device architectures, including iOS®, Android®, and other mobile device platforms. In some examples, the application may be developed to support other types of platforms, such as web-based or other computing platforms.

In some examples, the mobile application may be developed to run in the ‘background’—listening for push notifications to wake up the application. Such an architecture saves power on the mobile device, as compared with continuously polling the server for new messages. For push notifications to be delivered, access to a messaging or notification service compatible with the operating software of the mobile device may be provided and available, such as Google Cloud Messaging (GCM) for Android devices or Apple Push Notification Service (APNS) service for Apple devices. The application may utilize push notifications to alert the user of reminders to submit a survey response and/or other alerts, such as burnout mitigation activities or exercises.

The APNS servers may use load balancing, so devices may not always connect to the same public IP address for notifications. As such, it may be best to configure the application and backend servers to allow access to the aforementioned ports on the entire IP address block for APNS servers (e.g., the 17.0.0.0/8 address block).

As previously noted, Android push notifications may be powered by GCM. As such, any firewall that restricts the traffic to or from the Internet will need to allow connectivity to with GCM.

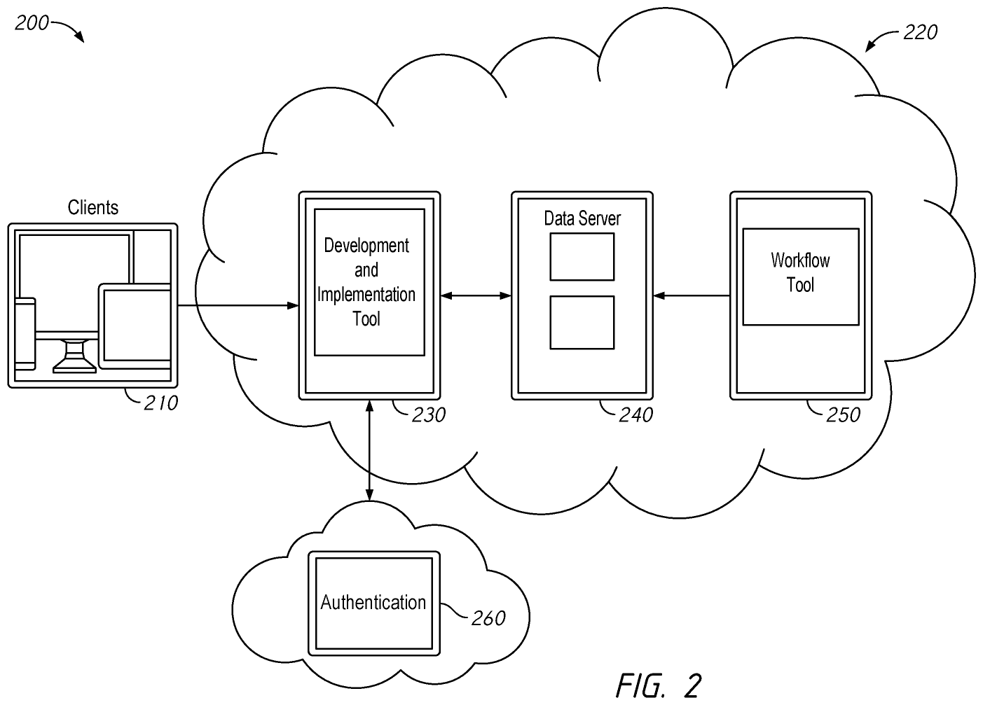

FIG. 2 depicts a block diagram of an exemplary system 200 to support the application hosted on the 210, in accordance with embodiments of the disclosure. With reference to FIG. 1, the system 200 may include a client 210 hosting an application that is configured to interface with a data center 220. In some examples, the data center 220 may be hosted in a cloud-based architecture. In other examples, the data center 220 may be hosted in a local server. The client 210 may include any mobile device or computer capable of hosting the application. It is appreciated that the system 200 may include multiple of the client 210 connected to the data center 220 without departing from the scope of the disclosure. The data center 220 may host components and data associated with the application running on the client 210, such as a 230, a 240, and a 250.

The 230 may provide a platform to support development and implementation of the application hosted on the client 210. The 240 may provide a platform to support storage of application data associated with and accessible to the application. That is, the application hosted on the client 210 may communication with the 240 to access application data relevant to the application. In some examples, the 240 may include a database. In some examples, the application may access the 240 using a secure protocol, such as HTTPS. In some examples, the application and/or the client 210 may implement additional, local security features to mitigate unauthorized access, such as a password, face identification, fingerprint identification, etc. For example, a 260 may implement an authentication protocol to authenticate users using the application via the client 210. The 250 may provide a platform to setup workflows to facilitate operation of various functions of the application, such as triggering a survey, presenting responses, storing data at the 240, etc.

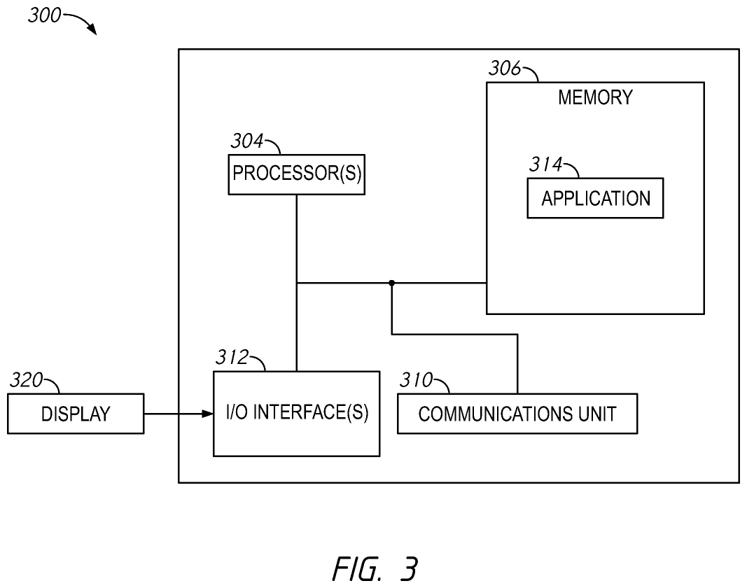

FIG. 3 depicts a block diagram of components of a computing device 300 in accordance with an embodiment of the present disclosure. It should be appreciated that FIG. 3 provides only an illustration of one implementation and does not imply any limitations with regard to the environments in which different embodiments may be implemented. Many modifications to the depicted environment may be made. The computing device 300 may implemented as the client 210 of FIG. 2, in some examples. In some examples, the computing device 300 may be implemented as any of the components of the data center 200 of FIG. 2. The computing device 300 may be configured to store executable instructions to perform methods described with reference to the burnout survey application described herein.

The computing device 300 includes a communications architecture to facilitate communication between one or more processor(s) 304, memory 306, communications unit 310, I/O interface(s) 312. The communications architecture can be implemented with any architecture designed for passing data and/or control information between processors (such as microprocessors, communications and network processors, etc.), system memory, peripheral devices, and any other hardware components within a system. For example, the communications architecture can be implemented with one or more buses.

The memory 306 are computer-readable storage media. In this embodiment, the memory 306 can include any suitable volatile or non-volatile computer-readable storage media. The memory 306 may be configured to store the application 314 in the form of executable instructions that when executed by the processor(s) 304, perform the methods directed to the burnout survey application described herein.

Communications unit 310, in these examples, provides for communications with other data processing systems or devices. In these examples, communications unit 310 includes one or more network interface cards. Communications unit 310 may provide communications through the use of either or both physical and wireless communications links.

I/O interface(s) 312 allows for input and output of data with other devices that may be connected to computing device 300. For example, I/O interface(s) 312 may provide a connection to external device(s) 318 such as a keyboard, a keypad, a touch screen, and/or some other suitable input device. External device(s) 318 can also include portable computer-readable storage media such as, for example, thumb drives, and memory cards. Software and data used to practice embodiments of the present disclosure can be stored on such portable computer-readable storage media and can be loaded onto local storage 308 via I/O interface(s) 312. I/O interface(s) 312 also connect to a display 320.

Display 320 provides a mechanism to display data to a user and may be, for example, a touchscreen.

### EXAMPLES

Example 1 is a mobile device, comprising: a memory configured to store instructions for a mood survey application; and a processor unit configured to execute the instructions for the mood survey application, wherein, when executed, the instructions cause the processor unit to: receive a request from a mood survey application database server to initiate a mood survey; in response to receipt of the notification, cause a notification to be provided to a user via a display including the request to perform the mood survey; in response to selection of the notification by the user, cause the mood survey to be presented to the user via the display; in response to completion of the survey by the user, provide mood survey results associated with the mood survey to the mood server database; and cause burnout mitigation resources received from the mood survey application database server to be presented to the user via the display, wherein the burnout mitigation resources are received based on the mood survey results.

In Example 2, the subject matter of Example 1 includes, wherein the instructions further cause the processor unit to provide a user-selected mood of a range of selectable moods of a mood scale presented during the survey with the mood survey results.

In Example 3, the subject matter of Example 2 includes, wherein the instructions further cause the processor unit to present the range of selectable moods of the mood scale to the user via the display during the mood survey.

In Example 4, the subject matter of Examples 2-3 includes, wherein the instructions further cause the processor unit to present the range of selectable moods as various emojis via the display during the mood survey.

In Example 5, the subject matter of Examples 2-4 includes, wherein the instructions further cause the processor unit to present the range of selectable moods as a number scale via the display during the mood survey.

In Example 6, the subject matter of Examples 2-5 includes, wherein the instructions further cause the processor unit to provide text comments received via selection by the user with the mood survey results.

In Example 7, the subject matter of Examples 1-6 includes, wherein the instructions further cause the processor unit to, in response to completion of the survey by the user, present data corresponding to recorded mood survey results of completed mood surveys associated with the user.

In Example 8, the subject matter of Example 7 includes, wherein the instructions further cause the processor unit to, in response to completion of the survey by the user, present the data corresponding to the recorded mood survey results of completed mood surveys associated with the user as a trend line showing change in mood survey results over time.

In Example 9, the subject matter of Examples 1-8 includes, wherein the instructions further cause the processor unit to present a mood journal page depicting a list of recorded mood survey results of completed mood surveys.

In Example 10, the subject matter of Examples 1-9 includes, wherein the instructions further cause the processor unit to present a selectable interface to start a new mood survey.

In Example 11, the subject matter of Examples 1-10 includes, wherein the instructions further cause the processor unit to receive subsequent requests from the mood survey application database server to initiate subsequent mood surveys at random intervals.

In Example 12, the subject matter of Examples 1-11 includes, wherein the instructions further cause the processor unit to receive subsequent requests from the mood survey application database server to initiate subsequent mood surveys at scheduled or periodic intervals.

Example 13 is at least one machine-readable medium including instructions that, when executed by processing circuitry, cause the processing circuitry to: receive, at an application hosted on a mobile device, a request from a mood survey application database server to initiate a mood survey; in response to receipt of the notification, display a notification on the display of the mobile device to notify the user of the request to perform the mood survey; in response to selection of the notification by the user, present the mood survey to the user via the display; in response to completion of the survey by the user, provide mood survey results associated with the mood survey to the mood survey application database server; and display burnout mitigation resources received from the mood survey application database server on the display, wherein the burnout mitigation resources are received based on the mood survey results.

In Example 14, the subject matter of Example 13 includes, wherein the instructions further cause the processing circuitry to provide a user-selected mood of a range of selectable moods of a mood scale presented during the survey with the mood survey results.

In Example 15, the subject matter of Example 14 includes, wherein the instructions further cause the processing circuitry to present the range of selectable moods of the mood scale to the user via the display during the mood survey.

In Example 16, the subject matter of Examples 14-15 includes, wherein the instructions further cause the processing circuitry to present the range of selectable moods as various emojis via the display during the mood survey.

In Example 17, the subject matter of Examples 14-16 includes, presenting the range of selectable moods as a number scale via the display during the mood survey.

In Example 18, the subject matter of Examples 14-17 includes, wherein the instructions further cause the processing circuitry to provide text comments received via selection by the user with the mood survey results.

In Example 19, the subject matter of Examples 13-18 includes, wherein the instructions further cause the processing circuitry to, in response to completion of the survey by the user, present data corresponding to recorded mood survey results of completed mood surveys associated with the user.

In Example 20, the subject matter of Example 19 includes, wherein the instructions further cause the processing circuitry to, in response to completion of the survey by the user, present the data corresponding to the recorded mood survey results of completed mood surveys associated with the user as a trend line showing change in mood survey results over time.

In Example 21, the subject matter of Examples 13-20 includes, wherein the instructions further cause the processing circuitry to present a mood journal page depicting a list of recorded mood survey results of completed mood surveys.

In Example 22, the subject matter of Examples 13-21 includes, wherein the instructions further cause the processing circuitry to present a selectable interface to start a new mood survey.

Example 23 is a method, comprising: receiving, at an application hosted on a mobile device, a request from a mood survey application database server to initiate a mood survey; in response to receipt of the notification, displaying a notification on the display of the mobile device to notify the user of the request to perform the mood survey; in response to selection of the notification by the user, presenting the mood survey to the user via the display; in response to completion of the survey by the user, providing mood survey results associated with the mood survey to the mood survey application database server; and displaying burnout mitigation resources received from the mood survey application database server on the display, wherein the burnout mitigation resources are received based on the mood survey results.

In Example 24, the subject matter of Example 23 includes, providing a user-selected mood of a range of selectable moods of a mood scale presented during the survey with the mood survey results.

In Example 25, the subject matter of Example 24 includes, presenting the range of selectable moods of the mood scale to the user via the display during the mood survey.

In Example 26, the subject matter of Examples 24-25 includes, presenting the range of selectable moods as various emojis via the display during the mood survey.

In Example 27, the subject matter of Examples 24-26 includes, presenting the range of selectable moods as a number scale via the display during the mood survey.

In Example 28, the subject matter of Examples 24-27 includes, providing text comments received via selection by the user with the mood survey results.

In Example 29, the subject matter of Examples 23-28 includes, in response to completion of the survey by the user, presenting data corresponding to recorded mood survey results of completed mood surveys associated with the user.

In Example 30, the subject matter of Example 29 includes, in response to completion of the survey by the user, presenting the data corresponding to the recorded mood survey results of completed mood surveys associated with the user as a trend line showing change in mood survey results over time.

In Example 31, the subject matter of Examples 23-30 includes, presenting a mood journal page depicting a list of recorded mood survey results of completed mood surveys.

In Example 32, the subject matter of Examples 23-31 includes, presenting a selectable interface to start a new mood survey.

Example 33 is at least one machine-readable medium including instructions that, when executed by processing circuitry, cause the processing circuitry to perform operations to implement of any of Examples 1-32.

Example 34 is an apparatus comprising means to implement of any of Examples 1-32.

Example 35 is a system to implement of any of Examples 1-32.

Example 36 is a method to implement of any of Examples 1-32.

While this disclosure describes implementations of the application in the context of a healthcare worker setting, it is appreciated that the application could be adapted or implemented to other types of settings, including other workplace or non-workplace organization settings.

Various illustrative components, blocks, configurations, modules, and steps have been described above generally in terms of their functionality. Persons having ordinary skill in the art may implement the described functionality in varying ways for each particular application, but such implementation decisions should not be interpreted as causing a departure from the scope of the present disclosure.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as previously described.

What is claimed is:

1. A mobile device, comprising:
a memory configured to store instructions for a mood survey application; and
a processor unit configured to execute the instructions for the mood survey application, wherein, when executed, the instructions cause the processor unit to:
receive a request from a mood survey application database server to initiate a mood survey;
in response to receipt of the notification, cause a notification to be provided to a user via a display including the request to perform the mood survey;
in response to selection of the notification by the user, cause the mood survey to be presented to the user via the display;
in response to completion of the survey by the user, provide mood survey results associated with the mood survey to the mood server database; and
cause burnout mitigation resources received from the mood survey application database server to be presented to the user via the display, wherein the burnout mitigation resources are received based on the mood survey results.

2. The mobile device of claim 1, wherein the instructions further cause the processor unit to provide a user-selected mood of a range of selectable moods of a mood scale presented during the survey with the mood survey results.

3. The mobile device of claim 2, wherein the instructions further cause the processor unit to present the range of selectable moods of the mood scale to the user via the display during the mood survey.

4. The mobile device of claim 2, wherein the instructions further cause the processor unit to provide text comments received via selection by the user with the mood survey results.

5. The mobile device of claim 1, wherein the instructions further cause the processor unit to, in response to completion of the survey by the user, present data corresponding to recorded mood survey results of completed mood surveys associated with the user.

6. The mobile device of claim 5, wherein the instructions further cause the processor unit to, in response to completion of the survey by the user, present the data corresponding to the recorded mood survey results of completed mood surveys associated with the user as a trend line showing change in mood survey results over time.

7. The mobile device of claim 1, wherein the instructions further cause the processor unit to present a mood journal page depicting a list of recorded mood survey results of completed mood surveys.

8. At least one machine-readable medium including instructions that, when executed by processing circuitry, cause the processing circuitry to:
receive, at an application hosted on a mobile device, a request from a mood survey application database server to initiate a mood survey;
in response to receipt of the notification, display a notification on the display of the mobile device to notify the user of the request to perform the mood survey;
in response to selection of the notification by the user, present the mood survey to the user via the display;
in response to completion of the survey by the user, provide mood survey results associated with the mood survey to the mood survey application database server; and
display burnout mitigation resources received from the mood survey application database server on the display, wherein the burnout mitigation resources are received based on the mood survey results.

9. The at least one machine-readable medium of claim 8, wherein the instructions further cause the processing circuitry to provide a user-selected mood of a range of selectable moods of a mood scale presented during the survey with the mood survey results.

10. The at least one machine-readable medium of claim 9, wherein the instructions further cause the processing circuitry to present the range of selectable moods of the mood scale to the user via the display during the mood survey.

11. The at least one machine-readable medium of claim 9 wherein the instructions further cause the processing circuitry to provide text comments received via selection by the user with the mood survey results.

12. The at least one machine-readable medium of claim 8, wherein the instructions further cause the processing circuitry to, in response to completion of the survey by the user, present data corresponding to recorded mood survey results of completed mood surveys associated with the user.

13. The at least one machine-readable medium of claim 12, wherein the instructions further cause the processing circuitry to, in response to completion of the survey by the user, present the data corresponding to the recorded mood survey results of completed mood surveys associated with the user as a trend line showing change in mood survey results over time.

14. A method, comprising:
receiving, at an application hosted on a mobile device, a request from a mood survey application database server to initiate a mood survey;
in response to receipt of the notification, displaying a notification on the display of the mobile device to notify the user of the request to perform the mood survey;
in response to selection of the notification by the user, presenting the mood survey to the user via the display;
in response to completion of the survey by the user, providing mood survey results associated with the mood survey to the mood survey application database server; and
displaying burnout mitigation resources received from the mood survey application database server on the display, wherein the burnout mitigation resources are received based on the mood survey results.

15. The method of claim 14, further comprising providing a user-selected mood of a range of selectable moods of a mood scale presented during the survey with the mood survey results.

16. The method of claim 15, further comprising presenting the range of selectable moods of the mood scale to the user via the display during the mood survey.

17. The method of claim 15, further comprising providing text comments received via selection by the user with the mood survey results.

18. The method of claim 14, further comprising, in response to completion of the survey by the user, presenting data corresponding to recorded mood survey results of completed mood surveys associated with the user.

19. The method of claim 18, further comprising, in response to completion of the survey by the user, presenting the data corresponding to the recorded mood survey results of completed mood surveys associated with the user as a trend line showing change in mood survey results over time.

20. The method of claim 14, further comprising presenting a mood journal page depicting a list of recorded mood survey results of completed mood surveys.
